(19) United States
(12) Patent Application Publication
(10) Pub. No.: US 2024/0009018 A1
(43) Pub. Date: Jan. 11, 2024

(54) Abdominal Wound Support Device

(71) Applicant: John Munson, Andover, MA (US)

(72) Inventor: John Munson, Andover, MA (US)

(21) Appl. No.: 17/858,201

(22) Filed: Jul. 6, 2022

(51) Int. Cl. A61F 5/30; A61F 5/03

(52) U.S. Cl. CPC A61F 5/30; A61F 5/03

(57) ABSTRACT

An abdominal wound support device is provided. The abdominal wound support device includes a frame. The frame includes a first end and a second end. A first pivotal member is placed on the first end of the frame. A second pivotal member is placed on the second end of the frame. A first handle is placed on the first pivotal member opposite the frame. A second handle is placed on the second pivotal member opposite the frame. A first roller is placed on the first pivotal member opposite the first handle. A second roller is placed on the second pivotal member opposite the second handle.

### BACKGROUND OF THE INVENTION

The present invention relates to an abdominal wound support device. More specifically, the present device provides an aid for post-operative patients who have undergone midline abdominal surgical processes.

Post-operative patients who have undergone midline abdominal surgical procedures often experience pain when coughing, sneezing, passing stools, or performing other sharp movements that strain the rectus abdominis muscles. This strain on the rectus muscles, which join at the midline, is caused by the contraction of the oblique abdominal muscles pulling laterally. The pain in an individual's post-operative wound can restrict the patient's ability to cough and clear secretions, leading to atelectasis, pneumonia, or other major post-operative complications. In extreme situations, the straining of these muscles can cause incisional hernias, which require corrective surgery.

Therefore, there is a need in the prior art for a device adapted to reduce the amount of strain placed on midline abdominal incisions. Furthermore, individuals who undergo surgical procedures are often forced to utilize narcotic medication to reduce the pain associated with the wounds caused by the surgical process. Therefore, there is also a need in the prior art for a device that reduces individuals' need to rely upon narcotic pain medication.

### SUMMARY OF THE INVENTION

In view of the foregoing disadvantages inherent in the known types of abdominal wound support therapies and apparatuses now present in the prior art, the present invention provides an abdominal wound support device wherein the same can be utilized for providing convenience for reducing strain for the user when recovering from a midline abdominal surgical procedure.

The present system comprises a frame. The frame comprises a first end and a second end. A first pivotal member is disposed on the first end of the frame. A second pivotal member is disposed on the second end of the frame. A first handle is disposed on the first pivotal member opposite the frame. A second handle is disposed on the second pivotal member opposite the frame. A first roller is disposed on the first pivotal member opposite the first handle. A second roller is disposed on the second pivotal member opposite the second handle.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the abdominal wound support device.

FIG. 2 shows a perspective view of an embodiment of the abdominal wound support device in use.

### DETAILED DESCRIPTION OF THE INVENTION

Reference is made herein to the attached drawings. Like reference numerals are used throughout the drawings to depict like or similar elements of the abdominal wound support device. The figures are intended for representative purposes only and should not be considered to be limiting in any respect.

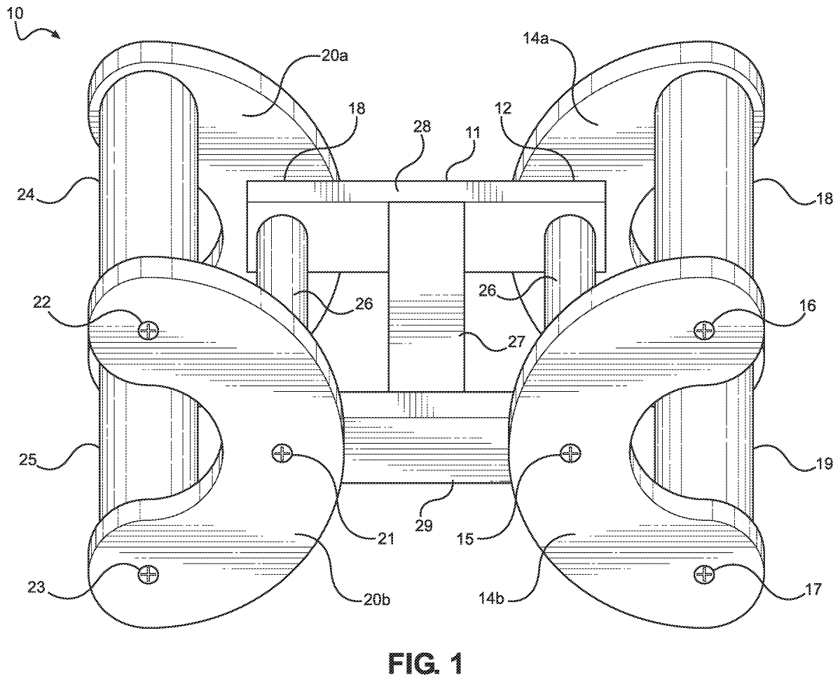

Referring now to FIG. 1, there is shown a perspective view of an embodiment of the abdominal wound support device. The abdominal wound support device 10 comprises a frame 11. The frame 11 comprises a first end 12 disposed opposite a second end 13. The frame 11 is configured to provide a base structure upon which additional elements may be secured. In the illustrated embodiment, the frame 11 comprises a central support 27 disposed between an upper support member 28 and a lower support member 29. The frame 11 is made of a rigid material, such as to reduce flexion of the frame 11. For example, the frame 11 may be made of plastic.

A first pivotal member 14a, 14b is disposed on the first end 12 of the frame 11. The first pivotal member 14a, 14b is pivotally affixed to the frame 11, such as to enable rotation of the first pivotal member 14a, 14b relative to the frame 11. In the illustrated embodiment, the first pivotal member 14a, 14b is arcuate in shape and defines an apex 15 at a medial point between a top end 16 and a lower end 17. Furthermore, in the illustrated embodiment, the first pivotal member 14a, 14b comprises a pair of symmetrical arcuate members consisting of a first symmetrical arcuate member 14a and a second symmetrical arcuate member 14b. The first pivotal member 14a, 14b may be pivotally secured to the frame 11 by any suitable hardware. For example, the first pivotal member 14a, 14b may be secured to the frame 11 via a cylindrical rod 26 disposed through a plurality of apertures through each of the first symmetrical arcuate member 14a, the frame 11, and the second symmetrical arcuate member 14b. The first pivotal member 14a, 14b is made of a rigid material, such as to reduce flexion of the first pivotal member 14a, 14b. For example, the first pivotal member 14a, 14b may be made of plastic.

A first handle 18 is disposed on the first pivotal member 14a, 14b at the top end 16 of the first pivotal member 14a, 14b. In the illustrated embodiment, the first handle 18 spans a distance between the first symmetrical arcuate member 14a and the second symmetrical arcuate member 14b. As such, a user may place his or her hand upon the first handle 18 to rotate the first pivotal member 14a, 14b relative to the frame 11. In one embodiment, the first handle 18 comprises a padded surface. As such, the user will more comfortably be able to grasp the first handle 18.

A first roller 19 is disposed on the first pivotal member 14a, 14b opposite the first handle 18. Specifically, in the illustrated embodiment, the first roller 19 is disposed at the lower end 17 of the first pivotal member 14a, 14b. The first roller 19 is configured to comfortably engage and apply inward pressure to the abdomen of the user when the first handle 18 is pulled downward. In some embodiments, the first roller 19 is rotatably affixed to the first pivotal member 14a, 14b such as to enable free rotation of the first roller 19. In the illustrated embodiment, the first roller 19 comprises a pad therearound. The pad is made of any suitable material for providing a softer surface when applying pressure to the patient's abdomen via the first roller 19. For example, the pad may be a foam pad.

A second pivotal member 20a, 20b is disposed on the second end 13 of the frame 11. The second pivotal member 20a, 20b is pivotally affixed to the frame 11, such as to enable rotation of the second pivotal member 20a, 20b relative to the frame 11. In the illustrated embodiment, the second pivotal member 20a, 20b is arcuate in shape and defines an apex 21 at a medial point between a top end 22 and a lower end 23. Furthermore, in the illustrated embodiment, the second pivotal member 20a, 20b comprises a pair of symmetrical arcuate members consisting of a first symmetrical arcuate member 20a and a second symmetrical arcuate member 20b. The second pivotal member 20a, 20b may be pivotally secured to the frame 11 by any suitable hardware. For example, the second pivotal member 20a, 20b may be secured to the frame 11 via a cylindrical rod 26 disposed through a plurality of apertures through each of the first symmetrical arcuate member 20a, the frame 11, and the second symmetrical arcuate member 20b. The second pivotal member 20a, 20b is made of a rigid material, such as to reduce flexion of the second pivotal member 20a, 20b. For example, the first pivotal member 20a, 20b may be made of plastic.

A second handle 24 is disposed on the second pivotal member 20a, 20b at the top end 22 of the second pivotal member 20a, 20b. In the illustrated embodiment, the second handle 24 spans a distance between the first symmetrical arcuate member 20a and the second symmetrical arcuate member 20b. As such, a user may place his or her hand upon the second handle 24 to rotate the second pivotal member 20a, 20b relative to the frame 11. In one embodiment, the second handle 24 comprises a padded surface. As such, the user will more comfortably be able to grasp the second handle 24.

A second roller 25 is disposed on the second pivotal member 20a, 20b opposite the second handle 24. Specifically, in the illustrated embodiment, the second roller 25 is disposed at the lower end 23 of the second pivotal member 20a, 20b. The second roller 25 is configured to comfortably engage and apply inward pressure to the abdomen of the user when the second handle 24 is pulled downward. In some embodiments, the second roller 24 is rotatably affixed to the second pivotal member 20a, 20b such as to enable free rotation of the second roller 24. In the illustrated embodiment, the second roller 25 comprises a pad therearound. The pad is made of any suitable material for providing a softer surface when applying pressure to the patient's abdomen via the second roller 25. For example, the pad may be a foam pad.

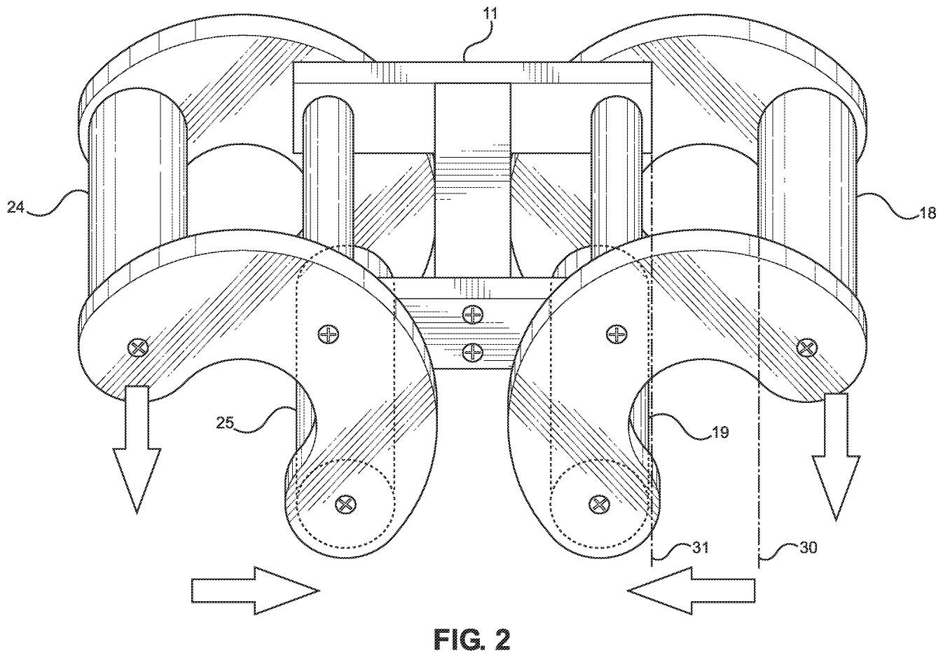

Referring now to FIG. 2, there is shown a perspective view of an embodiment of the abdominal wound support device in use. In use, the user 30 will place the abdominal wound support device 10 over his or her abdomen 31. The frame 11 will be placed over the midline of the abdomen 31, and the frame 11 may be utilized to position the abdominal wound support device 10. When the first handle 18 and the second handle 24 are pulled downward by the user 30, the first roller 19 and the second roller 25 are moved inward towards the midline of the abdomen 31, thereby applying inward pressure to the abdomen, pushing the rectus muscles towards the midline.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An abdominal wound support device, comprising:
a frame;
the frame comprising a first end disposed opposite a second end;
a first pivotal member disposed on the first end of the frame;
a second pivotal member disposed on the second end of the frame;
a first handle disposed on the first pivotal member at a first pivotal member top end;
a second handle disposed on the second pivotal member at a second pivotal member top end;
a first roller disposed on the first pivotal member opposite the first handle;
a second roller disposed on the second pivotal member opposite the second handle.

2. The abdominal wound support device of claim 1, wherein the first pivotal member and the second pivotal member are rotatably affixed to the frame via a cylindrical rod disposed between a pair of apertures.

3. The abdominal wound support device of claim 1, wherein the first handle and the second handle comprise a padded surface.

4. The abdominal wound support device of claim 1, wherein the frame comprises a central support disposed between an upper support member and a lower support member.

5. The abdominal wound support device of claim 1, wherein the first pivotal member and the second pivotal member comprise a pair of symmetrical arcuate members.

6. The abdominal wound support device of claim 1, wherein the first pivotal member and the second pivotal member are secured to the frame at an apex of each thereof.

7. The abdominal wound support device of claim 1, wherein the first roller and the second roller comprise a pad therearound.

8. The abdominal wound support device of claim 1, wherein the first roller and the second roller comprise a foam pad therearound.

9. The abdominal wound support device of claim 1, wherein the frame, the first pivotal member and the second pivotal member are composed of a rigid material.

10. The abdominal wound support device of claim 1, wherein the frame, the first pivotal member and the second pivotal member are composed of plastic.

11. The abdominal wound support device of claim 1, wherein the first roller is rotatably secured to the first pivotal member and the second roller is rotatably secured to the second pivotal member.
